(19) United States
(12) Patent Application Publication
(10) Pub. No.: US 2026/0237058 A1
(43) Pub. Date: Aug. 13, 2026

(54) METHOD AND SYSTEM FOR MANAGING MEDICAL IMAGE PROCESSING

(71) Applicant: Konica Minolta Healthcare Americas, Inc., Wayne, NJ (US)

(72) Inventor: Takafumi Nanjo, Gamer, NC (US)

(73) Assignee: Konica Minolta Healthcare Americas, Inc., Wayne, NJ (US)

(21) Appl. No.: 19/052,127

(22) Filed: Feb. 12, 2025

(51) Int. Cl. G06T 7/00; G16H 40/63

(52) U.S. Cl. CPC G06T 7/0012; G16H 40/63; G06T 2207/20081; G06T 2207/20084

(57) ABSTRACT

A method that includes receiving, by a server from a user device over a network, a medical image that is generated using a medical imaging device. The method further includes performing an image processing operation on the medical image based on an image processing parameter. The method further includes determining, by the server, whether the image processing operation is complete on the medical image. The method further includes transmitting, by the server to the user device over the network, a status message in response to determining that the first image processing operation is complete on the medical image.

### BACKGROUND

Medical images have been generated at hospitals for numerous medical applications, such as diagnosing patient symptoms as well as identifying the progress of medical treatments. Once a medical image is generated using a medical imaging technique, such as a computed tomography (CT) or magnetic resonance imaging (MRI), the medical image may follow several medical information protocols prior to being uploaded to a cloud environment. Once uploaded, medical images may be shared between group hospitals and medical personnel as well as used for remote image diagnosis and data backup.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In general, in one aspect, embodiments relate to a method that includes receiving, by a server from a user device over a network, a medical image that is generated using a medical imaging device. The method further includes performing an image processing operation on the medical image based on an image processing parameter. The method further includes determining, by the server, whether the image processing operation is complete on the medical image. The method further includes transmitting, by the server to the user device over the network, a status message in response to determining that the image processing operation is complete on the medical image.

In general, in one aspect, embodiments relate to a system that includes various user devices, a processing server, and a medical image manager coupled to the user devices and the processing server over a network. The medical image manager includes a computer processor. The medical image manager receives, from a user device among the user devices and over the network, a medical image that is generated using a medical imaging device. The medical image manager determines whether an image processing operation is complete on the medical image. The medical image manager transmits, to the user device over the network, a status message in response to determining that the image processing operation is complete on the medical image.

In general, in one aspect, embodiments relate to a nontransitory computer readable medium that includes instructions, when executed by a computer, are configured to perform a method. The method includes receiving, from a user device over a network, a medical image that is generated using a medical imaging device. The method further includes determining whether an image processing operation is complete on the medical image. The image processing operation is performed by a processing server. The method further includes transmitting, to the user device over the network, a status message in response to determining that the image processing operation is complete on the medical image.

In light of the structure and functions described above, embodiments of the invention may include respective means adapted to carry out various steps and functions defined above in accordance with one or more aspects and any one of the embodiments of one or more aspect described herein. Other aspects and advantages of the claimed subject matter will be apparent from the following description and the appended claims.

### BRIEF DESCRIPTION OF DRAWINGS

FIG. 1 shows a system in accordance with some embodiments.

FIG. 2 shows a flowchart in accordance with some embodiments.

FIGS. 3, 4, 5, 6, 7, 8A, and 8B show examples in accordance with some embodiments.

FIGS. 9A and 9B show a computing system in accordance with some embodiments.

### DETAILED DESCRIPTION

In the following detailed description of embodiments of the disclosure, numerous specific details are set forth in order to provide a more thorough understanding of the disclosure. However, it will be apparent to one of ordinary skill in the art that the disclosure may be practiced without these specific details. In other instances, well-known features have not been described in detail to avoid unnecessarily complicating the description.

Throughout the application, ordinal numbers (e.g., first, second, third, etc.) may be used as an adjective for an element (i.e., any noun in the application). The use of ordinal numbers is not to imply or create any particular ordering of the elements nor to limit any element to being only a single element unless expressly disclosed, such as using the terms “before”, “after”, “single”, and other such terminology. Rather, the use of ordinal numbers is to distinguish between the elements. By way of an example, a first element is distinct from a second element, and the first element may encompass more than one element and succeed (or precede) the second element in an ordering of elements.

In general, embodiments of the disclosure include systems and methods for managing image processing (IP) operations and/or artificial intelligence (AI) processing operations for medical images using various status messages. For example, a medical image manager may provide a network intermediary between one or more processing servers that perform IP and AI processing operations as well as user devices that upload medical images to a cloud. While previous prior art systems notified a user of whether a medical image had finished uploading to a particular network location, users did not receive any further update on the medical image's availability for further processing or use by a medical professional. With the advent of high-performance computing, some image and AI processes require a significant amount of computational time and computer resources to be performed within a cloud network. As such, one or more medical image managers may provide a smooth image diagnosis system by obtaining and reporting real-time information to users on the progress of specific processing in the cloud (e.g., whether a processing operation has begun, the amount of progress that has been completed, and whether the processing operation has terminated due to one or more processing errors).

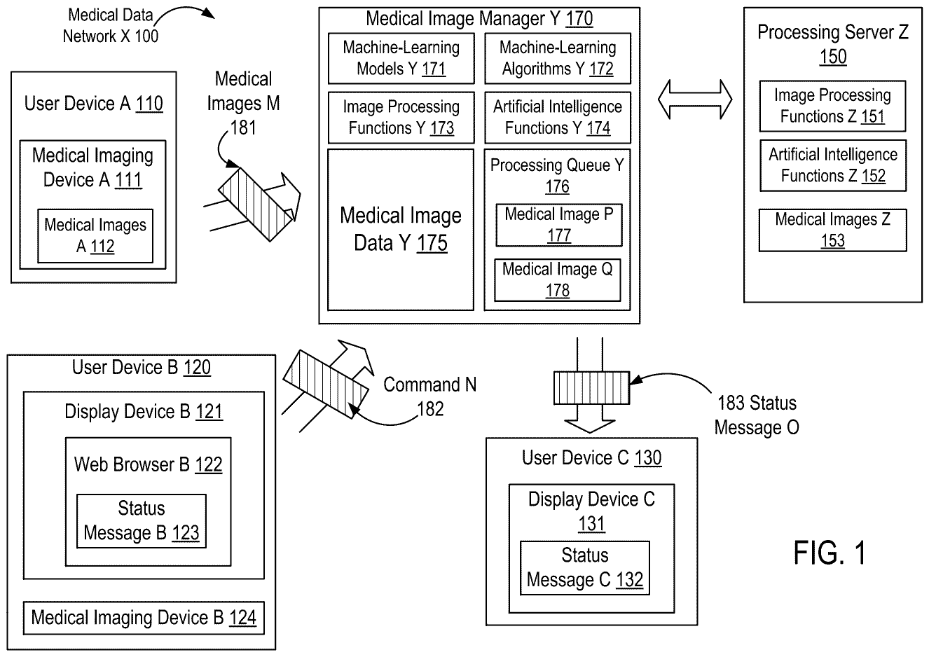

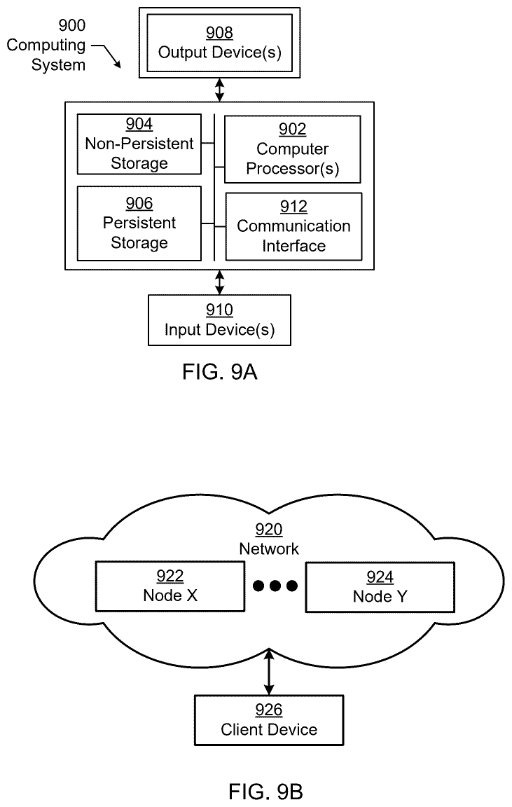

FIG. 1 shows a schematic diagram that includes a medical data network (e.g., medical data network X (100)) that may include various user devices (e.g., user device A (110), user device B (120), user device C (130)), and various servers (e.g., server Z (150)), various network elements (not shown), and a medical image manager (e.g., medical image manager Y (170)). User devices may include personal computers, smartphones, smart watches, human machine interfaces, hospital terminals (e.g., a workstation that allows medical professionals to access a hospital's Picture Archiving and Communication System (PACS)), and any other devices coupled to a computer network that obtain user inputs from users. The user devices may include an input device, a display device (e.g., display device B (121), display device C (131)), a communication interface, and hardware and/or software with functionality for providing a graphical user interface (GUI). A network element may refer to various hardware components within a computer network, such as switches, routers, and hubs, as well as user devices, servers, network storage devices, user equipment, or any other logical entities for uniting one or more physical devices on a computer network, such as the Internet. Medical image managers, user devices, processing servers, and network elements may include a computing system similar to the computing system (900) described in FIGS. 9A and 9B below.

A user device may be connected to one or more medical imaging devices (e.g., medical imaging device A (111), medical imaging device B (124)) for generating medical images (e.g., medical images A (112), medical image data Y (175), medical image P (177), medical image Q (178)). A medical imaging device may be an X-ray radiography device, an ultrasound device, or a device that performs magnetic resonance imaging (MRI), positron emission tomography (PET), computed tomography (CT) scans, or image processing workstation. For example, an ultrasound device may include hardware and/or software for emitting acoustic waves into a living subject and detecting reflected acoustic waves to generate an ultrasound image. These reflected acoustic waves may be analyzed to identify various properties of the subject's tissues through which one or more acoustic waves traveled, such as a density of the tissue. As such, an ultrasound device may include processing circuitry, input/output devices, ultrasound circuitry, and/or memory circuitry as well as other components such as transducer elements, a waveform generator, and a beamformer. On the other hand, an X-ray radiography device may include a radiation source, a radiation emission control device, a radiation detector, and a reading control device for viewing X-ray images generated using the radiation detector. The radiation source may include an X-ray tube that causes high-energy electrons to become accelerated and directed towards a target. Additionally, the radiation detector may include a semiconductor image sensor, such as a Flat Panel Detector (FPD) for producing X-ray images. For MRI devices, an MRI device may include a supporting structure (such as a vertical or horizontal wall) as well as a powerful magnet. Medical imaging devices may also include multi-modality devices such as an MRI-CT device and a PET-MRI device. Image processing workstations may include those that receive images from one or more modalities, verify the images or perform image processing (such as image reconstruction), and then send the images to Medical Image Manager.

In response to generating a medical image, a medical image file may be generated that associates the medical image with non-image content. For example, a medical image file may include various data fields, such as supplementary information which may include patient information, examination information, study information, series information, image-related information, and an image ID. Supplementary information may be written to the header of an image file as well as inserted as metadata into one or more embedded data fields. Series information may include information regarding a particular modality series (such as CT, MRI, DR, CR and US), a series instance UID, an examination portion, a series number, and a series description. The series instance UID may be a unique identifier (UID) for identifying a specific series. The series number is a number indicating the order of series in the same examination and may be used as a display order. Image-related information may include an image number, an image generation date, an imaging direction, an imaging start position, image type, and file path. The image number may refer to a number indicating the location of an image within the same series. The imaging start position may correspond to coordinates indicating a position at which an examination (e.g., a type of imaging scan) is started. The image type may include information indicating whether the image is a slice image or a scout image in the examination. The file path may refer to information indicating a storage location of the medical image in a particular database.

A medical imaging device or other medical device may use one or more medical data protocols for storing, transferring, and/or exchanging medical data. In particular, a medical image may be converted to a file format that conforms to the Digital Imaging and Communications in Medicine (DICOM) standard. A DICOM standard may include encrypting medical data into DICOM objects that include images and other information, such as demographic data, medical patient information, equipment data, examination details, basic image metadata, personal identifiable information (PII), and/or personal health information (PHI). Thus, a DICOM object may be used with a database to secure and keep confidential PII data and/or PHI data. As such, a DICOM protocol may enable communication and data exchange between different systems, such as a user device, a medical imaging device, a medical image manager, a telemedicine server, a cloud server, or a processing server, such as for performing IP operations or AI processing operations. DICOM objects may also include video clips or cine loops.

One or more IP functions (e.g., IP functions Y (173), IP functions Z (151)) may be applied to a medical image during an IP operation. For example, IP functions may include various techniques and algorithms that are applied to a digital image to analyze, enhance, and/or optimize image characteristics such as sharpness, contrast, size, and format. More specifically, an IP technique may treat a medical image as a data signal or a matrix for use with digital signal processing or matrix manipulation techniques, respectively. IP functions may be implemented using various parallel processing techniques, such as “general-purpose computing on graphics processing units” (GPGPU) in high-performance computing (HPC) servers and other multicore techniques.

Moreover, IP functions may include one or more compression techniques. Image compression may include lossy compression and lossless compression. While an image reconstructed by lossy compression is visually similar to the original image, the compressed image is not the same as the original image. On the other hand, a medical image reconstructed by lossless compression is precisely the same as the original medical image. Because patient information may be lost when using lossy compression, lossless compression is typically used for medical images. Examples of medical image compression include JPEG10 compression, JPEG-200011 compression, Huffman coding, arithmetic coding, Golomb coding, run length coding, Lempel-Ziv-Welch (LZW) coding, and predictive coding. Because a medical image may focus on a region of interest, some image compression functions apply lossless compression to the region of interest and lossy compression to the region of non-interest. In particular, JPEG10 compression uses a discrete cosine transform, while JPEG-200011 compression uses a wavelet transform. Other transform domains are also used in medical image compression, such as discrete orthogonal stockwell transforms and integer wavelet transforms.

Other IP functions may be specific to various modalities. For example, computed tomography includes various reconstruction algorithms that combine multiple projection images to create a 3D image. Examples of some CT reconstruction algorithms include iterative reconstruction (IR), filtered back projection (i.e., back-projecting an updated image onto itself until the differences between images reach a set value), weighted backprojections (WBPs) (i.e., reconstructing a 3D volume from 2D projections, and simultaneous iterative reconstruction techniques (SIRTs). Likewise, an MRI device may acquire raw MRI data that is not in image space. As such, an IP function may perform an image reconstruction process to produce an MRI image that can be interpreted clinically. For example, an MRI image reconstruction function may include noise pre-whitening for phased array data acquisition, interpolation between square pixels, raw data filtering for reducing Gibbs ringing artifacts, Fourier transforms to connect the raw MRI data with an image space, and phased array coil combinations.

IP operations may also include various X-ray imaging processes. For example, dynamic X-ray imaging may use one or more X-ray images to provide visualization and quantification of organ movements. As such, dynamic X-ray imaging may identify various lung diseases, such as chronic obstructive pulmonary disease (COPD), pneumonia, and lung cancer by providing new information of respiratory movements of the diaphragm, ribs, and other structures in the lung region. Moreover, dynamic X-ray imaging may be used in functional imaging of ventilation, blood circulation, and other bodily functions using various IP technologies. In contrast to static imaging, dynamic X-ray imaging may obtain additional information thus accelerating the elucidation of various pathologies, treatment starts, and assessment before and after therapy, and contributing to more efficient medical care and the reduction of medical costs. More specifically, dynamic X-ray imaging may use lung and heart recognition processing, time filtering processing, and reference frame subtraction processing to perform such IP.

Furthermore, a medical image manager (e.g., medical image manager Y (170)) may include hardware and/or software that deploys a medical system on a cloud network (e.g., medical data network X (100)). For example, a medical image manager may be a server that displays medical images using a network of servers, applications, and storage devices that are hosted on the Internet. In particular, a medical image manager may control the presentation and transmission of various medical image files to different medical personnel through various user devices (e.g., user device A (110), user device B (120), user device C (130)). One example of a medical image manager is a Picture Archiving and Communication System (PACS) that is used by various medical entities. Thus, a medical image manager may have hardware and/or software for storing, retrieving, managing, and distributing medical images to various user devices, medical devices, etc. In other words, the medical image manager may enable transmission of medical image data and other medical information (e.g., clinical reports and personal identifiable information for immediate use) between doctors, patients, medical experts, and external consultants. Thus, a medical image manager may securely transport private patient medical imaging information in contrast to retrieving physical files.

A medical image manager may further provide a smooth image diagnosis system to confirm whether medical images are uploaded to the cloud, and obtain real-time information on whether the IP and artificial intelligence processing is complete on the cloud service provider's side. In particular, a medical image manager may transmit various status messages (e.g., status message B (123), status message C (132), status message O (183)) over a computer network that are displayed in a graphical user interface (e.g., status message B (123) is displayed in a web browser B (122) on display device B (121)) on a user device or used by an application programmable interface. Status messages may indicate the state of various processing operations being performed with medical images in the cloud. Through automation of real-time status updates, various medical systems and medical professionals may achieve smooth inter-group collaboration and remote access to medical data. For example, a server (e.g., medical image manager or processing server) may perform server-side rendering, where the server performs rendering processes rather than the user device displaying medical data. Because rendering is performed at the server on medical images (e.g., medical images Z (153)), DICOM data may not be transmitted to the user device actually displaying a particular medical image and related information. Moreover, server-side rendering increases performance speed on the user device regardless of file size from newer acquisition modalities as well as minimize hardware technical requirements at the user device.

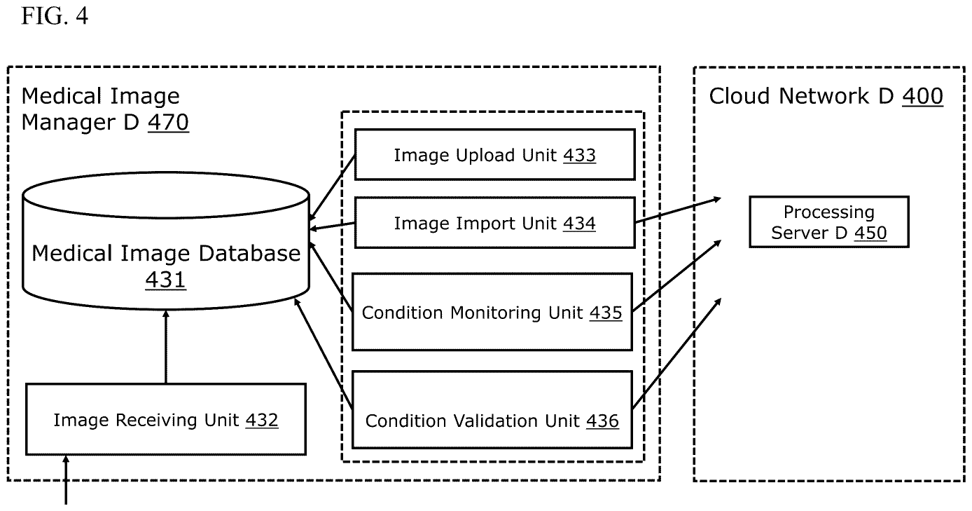

FIG. 4 shows an example of a medical image manager D (470) that includes a medical image database (431) connected to an image receiving unit (432), such as a communication interface for communicating with various network protocols. The medical image manager D (470) further includes an image upload unit (433), image import unit (434), a condition monitoring unit (435), and a condition validation unit (436). These various units may be various hardware and/or software modules that are configured to perform various functions with respect to processing server D (450) in a cloud network D (400).

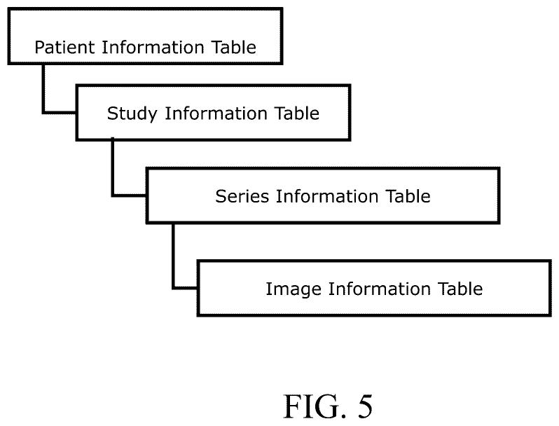

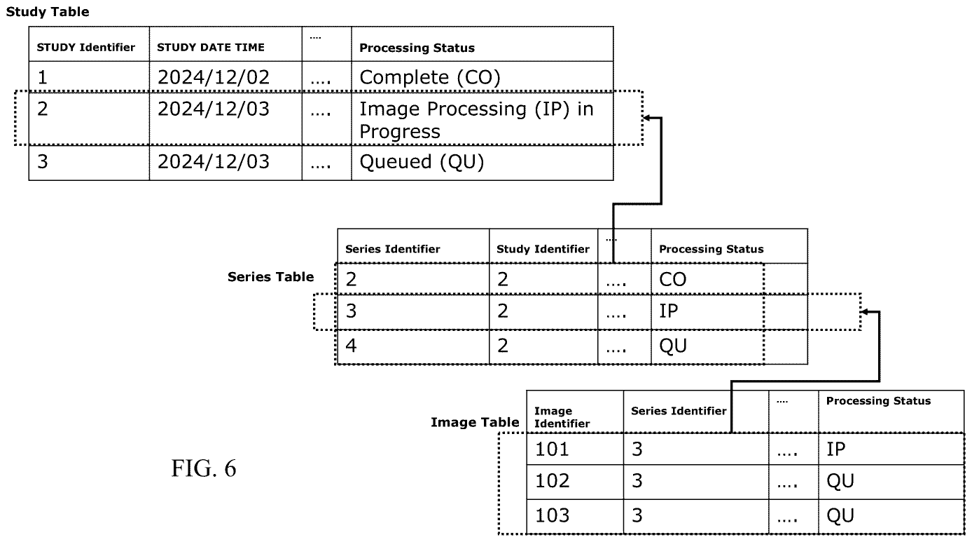

A medical image manager may further collect various types of medical data. For example, a medical image manager may store supplemental information alongside medical image data. Supplemental information may include one or more tables in a database, such as a patient table, a study table, a series tables, and an image table. For example, a medical image manager may have a database with the following table structure: Patient Information—Study Information—Series Information—Image Information. Status attributes of an IP operation and/or an AI processing operation may be maintained under the study information table at the medical image manager. For a parent table (if it is a series table, the study table is the parent), the parent table may be updated based on the information in the child tables (e.g., if all child tables statuses are “complete”, then the parent's records may also indicate “complete”). For example, status information regarding one or more medical images undergoing processing may be stored under the “study information” table. FIGS. 5 and 6 show examples of parent-child table relationships using this table structure.

Additionally, a medical image manager may transmit data (e.g., medical images M (181)) and/or commands (e.g., command N (182)) to one or more processing servers (e.g., processing server Z (150)). For example, a processing server may be used to provide one or more cloud-native medical imaging applications. The processing server may import data in DICOM P10 format from the medical image manager and provide application programmable interfaces (APIs) for low latency retrieval and dedicated storage. When a processing server is performing an import operation, the medical image manager may receive various status messages, such as whether a medical image has been submitted to the processing server, whether a particular processing operation is in progress, whether the processing operation is completed, whether a medical image has been validated, and whether a processing operation has failed or otherwise experienced an error.

Various queues (e.g., processing queue Y (176)) may be used to manage uploading and/or processing of various medical images. For example, a processing queue may be used to manage uploading multiple medical image files to a network location within a cloud network. Likewise, a processing queue may also determine a priority of different medical images that are under an IP operation and/or an AI processing operation. Processing queues may also be following various queue algorithms, such as first-in-first-out to determine a sequence of medical image processing.

A medical image manager and/or user device may adjust processing performed using commands. Examples of commands include network messages that are transmitted over a machine-to-machine network protocol, or a control signal received that automatically triggering one or more server operations. A command may correspond to a request to perform a particular processing operation or a request for status information on one or more scheduled processing operations. Likewise, a command may terminate or adjust a processing operation in real-time (e.g., by changing image processing parameters or AI parameters).

A medical image manager may include hardware and/or software with functionality for performing various AI functions (e.g., AI functions Y (174), AI functions Z (152)). For example, AI functions may be used for different AI processing operations, such as generating and/or updating a machine-learning model (e.g., machine-learning models Y (171)) as well as implementing inference operations. For example, a machine-learning model may obtain as inputs one or more medical images as well as other information (e.g., patient information, study information, series information, etc.). These inputs may be used by the machine-learning model for outputting a predicted diagnosis or other medical information (e.g., the boundaries of a particular organ within an X-ray, CT scan, or MRI scan). Additionally, different types of machine-learning models may be trained, such as convolutional neural networks, deep neural networks, U-net models, recurrent neural networks, support vector machines, decision trees, inductive learning models, deductive learning models, supervised learning models, unsupervised learning models, reinforcement learning models, etc. In a deep neural network, for example, a layer of neurons may be trained on a predetermined list of features based on the previous network layer's output. Thus, as data progresses through the deep neural network, more complex features may be identified within the data by neurons in later layers. Moreover, two or more different types of machine-learning models are integrated into a single machine-learning architecture, e.g., a machine-learning model may include decision trees and neural networks. In some embodiments, a medical image manager or processing server may generate augmented or synthetic data to produce a large amount of interpreted data for training a particular model.

With respect to support vector machines, a support vector machines may be a machine-learning model that is trained using a supervised machine-learning algorithm. For example, a support vector machine may provide a data analysis on various input features that implement a classification and regression analysis. More specifically, a support vector machine may determine a hyperplane that separates a dataset into different classes, and also determines various points (i.e., support vectors) that lie closest to different classes. Additionally, a support vector machine may use one or more kernel functions to transform data into a desired form for further processing. The term “kernel” may refer to a set of mathematical functions that provide the window to manipulate the input data. In other words, a kernel function may transform a training set of data so that a non-linear decision surface is able to transform to a linear equation into a higher number of dimension spaces. Examples of kernel functions may include gaussian kernel functions, gaussian kernel radial basis functions (RBFs), sigmoid kernel functions, polynomial kernel functions, and linear kernel functions.

With respect to artificial neural networks, for example, a neural network may include one or more hidden layers, where a hidden layer includes one or more neurons. A neuron may be a modelling node or object that is loosely patterned on a neuron of the human brain. In particular, a neuron may combine data inputs with a set of coefficients, i.e., a set of network weights for adjusting the data inputs. These network weights may amplify or reduce the value of a particular data input, thereby assigning an amount of significance to various data inputs for a task being modeled. Through machine learning, a neural network may determine which data inputs should receive greater priority in determining one or more specified outputs of the neural network. Likewise, these weighted data inputs may be summed such that this sum is communicated through a neuron's activation function to other hidden layers within the neural network. As such, the activation function may determine whether and to what extent an output of a neuron progresses to other neurons where the output may be weighted again for use as an input to the next hidden layer.

Turning to convolutional neural networks, a convolutional neural network (CNN) is a type of artificial neural network that may be used in computer vision and image recognition, e.g., for processing pixel data. For example, a convolutional neural network may include functionality for performing an application of a filter to an input (e.g., an input image) that results in a particular activation, where repeated filter application may result in an output map of activations called a feature map. A feature map may indicate the locations and strength of one or more detected features in the input to the convolutional neural network. Thus, a convolutional neural network may have the ability to automatically learn multiple filters in parallel specific to a training dataset under the constraints of a specific predictive modeling problem, such as image classification.

In some embodiments, a server uses one or more ensemble learning methods in connection to various machine-learning models. For example, an ensemble learning method may use multiple types of machine-learning models to obtain better predictive performance than available with a single machine-learning model. In some embodiments, for example, an ensemble architecture may combine multiple base models to produce a single machine-learning model. One example of an ensemble learning method is a BAGGing model (i.e., BAGGing refers to a model that performs Bootstrapping and Aggregation operations) that combines predictions from multiple neural networks to add a bias that reduces variance of a single trained neural network model. Another ensemble learning method includes a stacking method, which may involve fitting many different model types on the same data and using another machine-learning model to combine various predictions. In some embodiments, two or more different types of machine-learning models are integrated into a single machine-learning architecture, e.g., a machine-learning model may include support vector machines and neural networks.

In some embodiments, various types of machine-learning algorithms (e.g., machine-learning algorithm Y (172)) may be used to train the model, such as a backpropagation algorithm. In a backpropagation algorithm, gradients are computed for each hidden layer of a neural network in reverse from the layer closest to the output layer proceeding to the layer closest to the input layer. As such, a gradient may be calculated using the transpose of the weights of a respective hidden layer based on an error function (also called a “loss function”). The error function may be based on various criteria, such as mean squared error function, a similarity function, etc., where the error function may be used as a feedback mechanism for tuning weights in the machine-learning model.

In some embodiments, a machine-learning model is trained using multiple epochs. For example, an epoch may be an iteration of a model through a portion or all of a training dataset. As such, a single machine-learning epoch may correspond to a specific batch of training data, where the training data is divided into multiple batches for multiple epochs. Thus, a machine-learning model may be trained iteratively using epochs until the model achieves a predetermined criterion, such as predetermined level of prediction accuracy or training over a specific number of machine-learning epochs or iterations. Thus, better training of a model may lead to better predictions by a trained model.

Turning to recurrent neural networks, a recurrent neural network (RNN) may perform a particular task repeatedly for multiple data elements in an input sequence, with the output of the recurrent neural network being dependent on past computations. As such, a recurrent neural network may operate with a memory or hidden cell state, which provides information for use by the current cell computation with respect to the current data input. For example, a recurrent neural network may resemble a chain-like structure of RNN cells, where different types of recurrent neural networks may have different types of repeating RNN cells. Likewise, the input sequence may be time-series data, where hidden cell states may have different values at different time steps during a prediction or training operation. For example, where a deep neural network may use different parameters at each hidden layer, a recurrent neural network may have common parameters in an RNN cell, which may be performed across multiple time steps. To train a recurrent neural network, a supervised learning algorithm such as a backpropagation algorithm may also be used. In some embodiments, the backpropagation algorithm is a backpropagation through time (BPTT) algorithm. Likewise, a BPTT algorithm may determine gradients to update various hidden layers and neurons within a recurrent neural network in a similar manner as used to train various deep neural networks. In some embodiments, a recurrent neural network is trained using a reinforcement learning algorithm such as a deep reinforcement learning algorithm. For more information on reinforcement learning algorithms, see the discussion below.

Embodiments are contemplated with different types of RNNs. For example, classic RNNs, long short-term memory (LSTM) networks, a gated recurrent unit (GRU), a stacked LSTM that includes multiple hidden LSTM layers (i.e., each LSTM layer includes multiple RNN cells), recurrent neural networks with attention (i.e., the machine-learning model may focus attention on specific elements in an input sequence), bidirectional recurrent neural networks (e.g., a machine-learning model that may be trained in both time directions simultaneously, with separate hidden layers, such as forward layers and backward layers), as well as multidimensional LSTM networks, graph recurrent neural networks, grid recurrent neural networks, etc. With regard to LSTM networks, an LSTM cell may include various output lines that carry vectors of information, e.g., from the output of one LSTM cell to the input of another LSTM cell. Thus, an LSTM cell may include multiple hidden layers as well as various pointwise operation units that perform computations such as vector addition.

With respect to region-based convolutional neural networks, a region-based convolutional neural network (R-CNN) may obtain an input image or other image data at an input layer. The R-CNN may then perform a selective search function to extract various regions of interest (ROIs), where an ROI may correspond to a predetermined boundary (e.g., such as a particular rectangle) of an object in the input image. For example, an input petrographic image may include a thousand regions of interest (or region proposals) that are being analyzed by the R-CNN. After determining the image data for different regions, respective image data for respective regions may be sent through a neural network to determine various output features, such as whether a particular region proposal corresponds to a heart object or non-heart object. For each region's output features, a collection of support vector machines may operate as classifiers that may be used to determine what type of object is contained within the respective region. Moreover, various regions that are used by an R-CNN may be referred to as ‘region proposals’ that identify smaller regions of image data that possibly include objects being searched for in the input image data. To reduce the region proposals in the R-CNN, a selective search function may be used accordingly. Moreover, the convolutional neural network and the support vector machines may be trained separately based on their classifying function within the R-CNN.

Furthermore, various types of region-based convolutional neural networks are contemplated. For example, an R-CNN may be a Fast R-CNN, a Faster R-CNN, a Mask R-CNN, or a you-only-look-once (YOLO) network. While a regular R-CNN may independently determine neural network features on each region of interest, a Fast R-CNN may use a neural network only once on an entire image. At the end of the convolutional neural network, an ROI Pooling process may be performed, which slices out each region from the network's output tensor, reshapes the output features, and subsequently classifies the reshaped output features, such as to determine lithology parameters or cutting parameters. As in a regular R-CNN, the Fast R-CNN may also use a selective search function or process to generate various region proposals. In a Faster R-CNN, the Faster R-CNN may integrate the ROI generation into the convolutional neural network itself. In a YOLO network, the YOLO network may perform similarly to a fully convolutional neural network, by passing the image once through the FCNN and output a particular prediction for a grid that includes bounding boxes and class probabilities for the bounding boxes. In a Mask R-CNN, the Mask R-CNN may include object instance segmentation. Object Instance Segmentation may detect object classes (e.g., whether a portion of medical image data is a bone or another organ) along with determining a segmenting of a mask for each object instance. Likewise, some machine-learning models are contemplated that perform only semantic segmentation, such as distinguishing between biological objects within medical image data, or detecting the presence of other image object types.

An example of an AI function may be a positioning judgment function that identifies an area and extent of displacement of a joint on a medical image. The positioning judgment function may be an inference function that identifies a local area of a joint within an image based on machine-learning model (e.g., using a deep neural network). The positioning judgment function may further perform segmentation processing of the identified local area based on machine learning to identify the displaced area. As such, the positioning judgment function may measure the distance of the segmented area from the center of the joint to determine a maximally displaced width.

Another example of an AI function includes a left/right judgment function that is another inference function that firstly performs pretreatment to change an image size and adjust a gradation and density of the captured medical image. The left/right judgment function may estimate a probability that a medical image is that of the left side or the right side of a joint based on machine learning. The left/right judgment function may also determine the image side with the higher probability as the side of the joint of which the medical image was actually taken. These AI functions may involve high-speed processing because the output results may require immediate display on a user device after the X-ray images have been acquired.

While FIG. 1 shows various configurations of components, other configurations may be used without departing from the scope of the disclosure. For example, various components in FIG. 1 may be combined to create a single component. As another example, the functionality performed by a single component may be performed by two or more components.

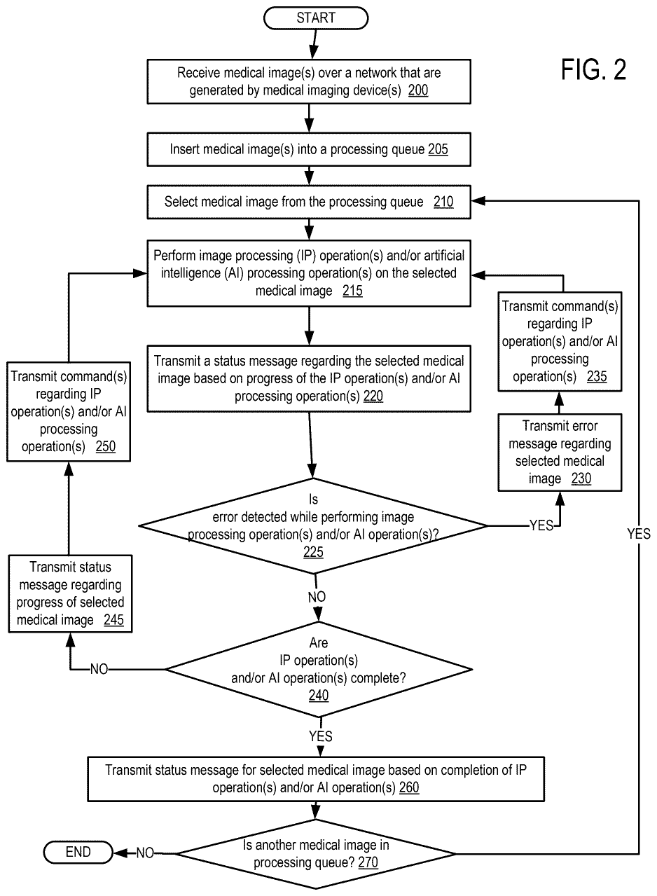

FIG. 2 describes a general method for providing status messages regarding various processing operations. Blocks in FIG. 2 may be performed by various components (e.g., medical image manager Y (170) or a processing server Z (150)) as described in FIG. 1. While the various blocks in FIG. 2 are presented and described sequentially, one of ordinary skill in the art will appreciate that some or all of the blocks may be executed in different orders, may be combined or omitted, and some or all of the blocks may be executed in parallel. Furthermore, the blocks may be performed actively or passively.

In Block 200, one or more medical images are received over a network that are generated by one or more medical imaging devices. For example, a medical imaging device may be an X-ray radiography device, an MRI device, or other imaging device that is connected to a user device. After acquiring the medical images using one or more medical imaging devices, the medical images may be uploaded to a medical image manager or other server. On the other hand, a user device may also acquire one or more medical images from a network, such as a local database, and upload the medical images accordingly.

In Block 205, one or more medical images are inserted into a processing queue. For example, a processing queue may be an upload queue located at a user device or a medical image manager that determines a sequence that medical images are uploaded to a particular network location. Additionally, a processing queue may identify the sequence that different medical images undergo IP operations and/or AI processing operations. As such, a medical image may also be inserted into a processing queue on a server that performs IP and/or AI processing.

In Block 210, a medical image is selected from a processing queue. For example, various medical images may be selected iteratively by a medical image manager according to a predetermined order that various image files are uploaded to a processing server. Likewise, a medical image may be selected for further processing based on a user selection within a graphical user interface or automatically by a medical image manager based on a network request.

In Block 215, one or more IP operations and/or one or more artificial intelligence (AI) processing operations are performed on a selected medical image. For example, an IP operation and/or an AI processing operation may be selected by a user in connection to uploading the selected image. Likewise, various processing operations may be performed automatically in response to uploading an image. For example, medical images may automatically undergo one or more types of image compression prior to making the medical image available for clinical usage at one or more user devices.

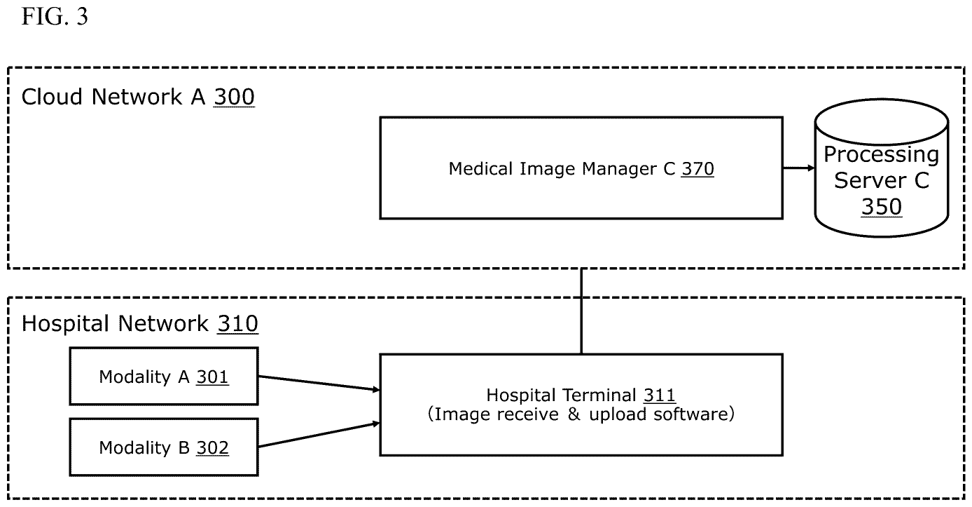

FIG. 3 shows an example where a hospital terminal (311) on a hospital network (310) communicates with medical image manager C (370) on a cloud network C (300). The hospital terminal (311) obtains images for different modalities, i.e., modality A (301) and modality B (302), that are transmitted to the medical image manager C (370). Afterwards, the medical image manager relays the medical images to a processing server C (350) for an IP operation and/or AI processing operation. Some examples of modalities may include a type of treatment method, a type of medical device (e.g., medical imaging device), or a strategy type used to treat a patient's condition or injury. Modalities may include physical, electrical, mechanical, or chemical modalities.

In Block 220, a status message is transmitted regarding a selected medical image based on progress of one or more IP operations and/or one or more AI processing operations. In particular, a medical image may be identified within a graphical user interface as having a status attribute of “QUEUE,” “IN PROGRESS,” “ERROR,” “COMPLETE,” “IP,” and “ARTIFICIAL INTELLIGENCE PROCESSING.” The “QUEUE” status may identify a medical image before uploading and/or before further progressing. “IN PROGRESS” may identify a medical image being uploaded, undergoing IP, and/or AI processing. The “ERROR” status may correspond to an error related to uploading, IP, and/or AI processing. Moreover, some status message may specify the related processing, such as an “IP ERROR” or an “AI ERROR” may correspond to an IP error and/or an AI processing error, respectively. A status message may also specify whether particular processing is complete, such as “IP COMPLETE,” “AI COMPLETE,” or “UPLOAD COMPLETE.”

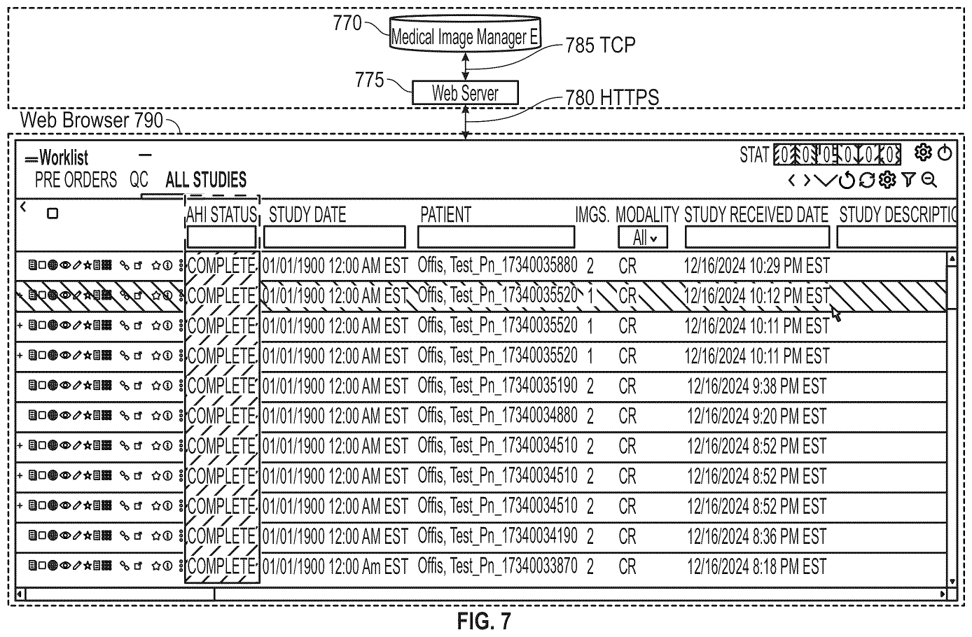

Furthermore, various status messages may be displayed on a user device, such as in a web browser or as a notification from a medical image manager. FIG. 7 shows an example where a medical image manager E (770) communicates with a web server (775) using a transmission control protocol (TCP) (785). The web server (775) then communicates a user device (not shown) with Hypertext Transfer Protocol Secure (HTTPS) (780) to transmit status messages displayed in a web browser (790). Thus, a user may communicate with a medical image manager through a web browser, where FIG. 7 shows a display screen called Study Worklist, which allows the user to view information for a study base. On this list, the user can see the status of various medical images on a processing server.

In Block 225, a determination is made whether an error is detected while performing one or more IP operations and/or one or more AI processing operations. For example, an error message may be outputted by an IP function and/or an AI function indicating an error during processing. Likewise, an image output from an IP or AI processing operation may be validated using one or more error detection functions by a medical image manager or processing server to determine whether one or more errors occurred during processing. For a compression operation, an error detection function may compare a compressed image with the original medical image to determine the presence of one or more errors. Likewise, testing data may be used with a trained machine-learning model to verify whether the model achieves a desired level of accuracy.

In Block 230, an error message is transmitted regarding a selected image. For example, a processing server may relay an error message to a medical image manager. Afterwards, the medical image manager may transmit a status message indicating one or more errors to one or more user devices over a network.

In Block 235, one or more commands are transmitted regarding one or more IP operations and/or one or more AI processing operations. For example, a user device may receive a user selection of one or more commands in a graphical user interface in response to receiving a status message indicating one or more errors. More specifically, a user may change one or more image parameters of an IP operation and/or one or more AI parameters of an AI processing operation to resolve an error. For illustration, a compression process may be selected based on a particularity modality for a medical image that is incorrect. In reviewing the error message, a different type of compression may be selected for the modality used to obtain the uploaded medical image. In particular, a user device may transmit a request to a medical image manager or processing server that adjusts the corresponding image parameters of the IP operation. Once the request is received, an adjusted IP operation may automatically be restarted using the adjusted parameters.

For AI processing operations, a user device may transmit different commands to change the type of inference operation as well as various AI parameters associated with a respective AI processing operation. For example, a user may select a different machine-learning model in response to a particular error. If an error occurs during a training operation (such as the model never achieves a desired level of prediction accuracy), a user may adjust the operation to use a different training dataset, a different initial model, and various hyperparameters associate with the trained model. For inference operations, a user may select a different inference operation being performed on a selected medical image (e.g., an inference operation for diagnosing heart disease may produce an error when using a medical image of a broken femur). As such, a user may analyze a selected medical image undergoing AI processing using one or more error detection functions to determine whether to change the type of inference operation.

In Block 240, a determination is made whether one or more IP operations and/or one or more AI processing operations are complete. In response to performing an IP operation and/or AI processing operation, a user device may display a status message indicating the respective operation is finished. Afterwards, one or more user devices may access the output data associated with the particular processing operation. If a medical is compressed, a doctor or other medical expert may access the compressed image for faster access. If an inference operation is performed, a user may access predicted data acquired by the inference operation. If a training operation is performed, the trained model may become available for predicting patient data after completion.

In Block 245, status message is transmitted regarding progress of selected medical image. For example, the status message may simply indicate whether a specific IP operation or AI processing operation is still being performed. On the other hand, the status message may also indicate a degree of completion (e.g., 70% complete) for a particular operation.

In Block 250, one or more commands are transmitted regarding one or more IP operations and/or one or more AI processing operations. For example, a user may decide to change a type of IP operation and/or AI processing operation prior to completion. Accordingly, a user device may transmit a command to terminate an ongoing processing operation, such as to use a different selected image for the processing operation or change the type of processing operation (e.g., adjust the parameters of an IP operation).

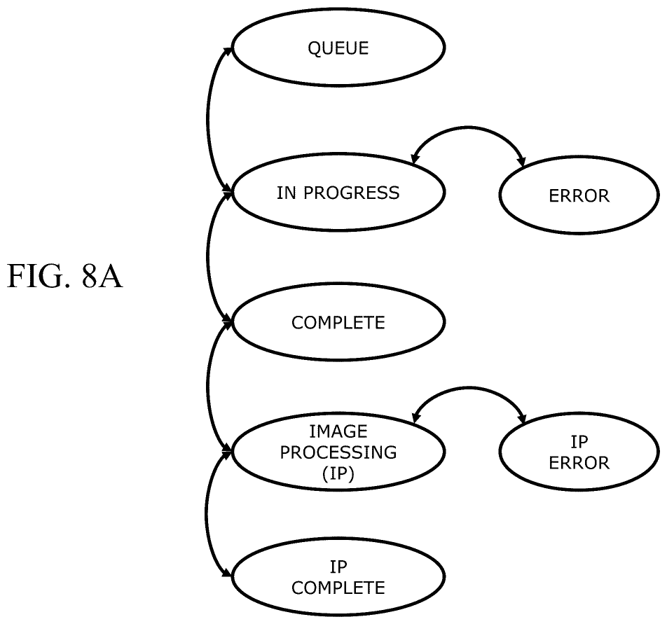

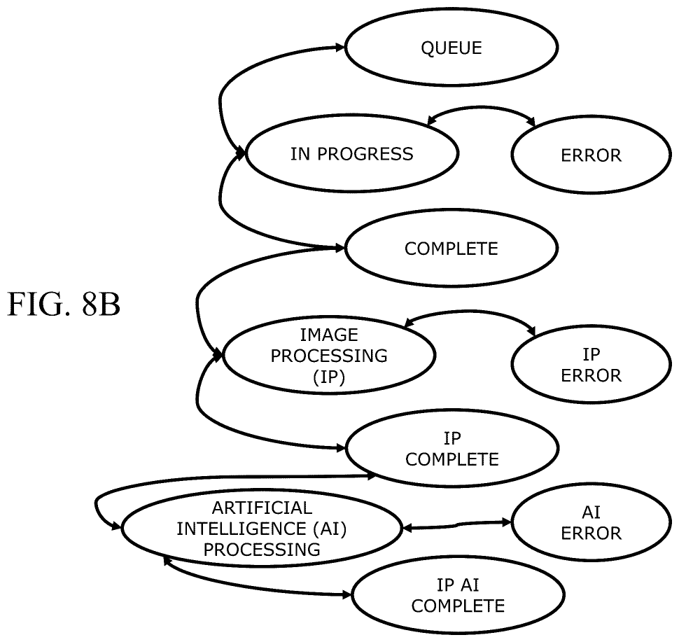

In Block 260, status message is transmitted for a selected medical image based on completion of one or more IP operations and/or one or more AI processing operations. Once a medical image manager determines that an IP operation or AI processing operation is complete, the medical image manager may transmit a status message to a user device to indicate the process's completion. As such, the selected medical image may be made further available to various users for access as well as further processing (e.g., after an IP operation is complete, one or more AI processing operations may be further performed on the selected image to determine medical diagnostic information for a clinician). FIG. 8A shows an example decision tree of a medical image go through various possible status messages based on a queue and an IP operation. FIG. 8B shows an example decision tree based on traversing an IP operation and an AI processing operation.

In Block 270, a determination is made whether another medical image is disposed in a processing queue. For example, a medical image manager or a processing server may proceed through a particular queue until all IP operations and/or AI processing operations have been performed. If another medical image is disposed in the processing queue, Blocks 210-260 may be repeated.

FIG. 9A shows a computing system (900) that may include any combination of mobile, desktop, server, router, switch, embedded device, or other types of hardware. The computing system (900) may include one or more computer processors (902), non-persistent storage (904) (e.g., volatile memory, such as random access memory (RAM), cache memory), persistent storage (906) (e.g., a hard disk, an optical drive such as a compact disk (CD) drive or digital versatile disk (DVD) drive, a flash memory, etc.), and a communication interface (912) (e.g., Bluetooth interface, infrared interface, network interface, optical interface, etc.). The computer processor(s) (902) may be an integrated circuit for processing instructions. For example, the computer processor(s) may be one or more cores or micro-cores of a processor. The computing system (900) may also include one or more input devices (910), such as a touchscreen, keyboard, mouse, microphone, touchpad, electronic pen, or any other type of input device. The communication interface (912) may include an integrated circuit for connecting the computing system (900) to a network (e.g., a local area network (LAN) or a wide area network (WAN) such as the Internet or cellular network).

Further, the computing system (900) may include one or more output devices (908), such as a screen (e.g., a liquid crystal display (LCD), a plasma display, touchscreen, projector, or other display device), a printer, external storage, or any other output device. One or more of the output devices may be the same or different from the input device(s). The computing system (900) may implement and/or be connected to a data repository. For example, one type of data repository is a database. A database is a collection of information configured for ease of data retrieval, modification, re-organization, and deletion. Database Management System (DBMS) is a software application that provides an interface for users to define, create, query, update, or administer databases.

Software instructions in the form of computer readable program code to perform various functions may be stored, in whole or in part, temporarily or permanently, on a non-transitory computer readable medium such as a storage device, a diskette, a tape, flash memory, physical memory, or any other computer readable storage medium. Specifically, the software instructions may correspond to computer readable program code that, when executed by a processor(s), is configured to perform various functions.

The computing system (900) in FIG. 9A may be connected to or be a part of a network. For example, as shown in FIG. 9B, the network (920) may include multiple nodes (e.g., node X (922), node Y (924)). Each node may correspond to a computing system, such as the computing system shown in FIG. 9A, or a group of nodes combined may correspond to the computing system shown in FIG. 9A. The nodes (e.g., node X (922), node Y (924)) in the network (920) may be configured to provide services for a client device (926). The nodes may include functionality to receive requests from the client device (926) and transmit responses to the client device (926). The client device (926) may be a computing system, such as the computing system shown in FIG. 9A.

The computing system (900) may be implemented as part of a cloud computing system. For example, the nodes may be part of a cloud computing system. For example, a cloud computing system may include remote servers along with various other cloud components, such as cloud storage units and edge servers. As such, a cloud computing system may have different functions distributed over multiple locations from a central server, which may be performed using Internet connections.

The computing system may further include functionality to receive data from a user. For example, a user may submit data via a graphical user interface (GUI) on the user device. Data may be submitted via the graphical user interface by a user selecting one or more graphical user interface components or inserting text and other data into graphical user interface widgets using a touchpad, a keyboard, a mouse, or any other input device. In response to selecting a particular item, information regarding the particular item may be obtained from persistent or non-persistent storage by the computer processor. Upon selection of the item by the user, the contents of the obtained data regarding the particular item may be displayed on the user device in response to the user's selection.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method, comprising:
receiving, by a server from a user device over a network, a first medical image that is generated using a medical imaging device;
performing a first image processing operation on the first medical image based on at least one image processing parameter;
determining, by the server, whether the first image processing operation is complete on the first medical image; and
transmitting, by the server to the user device over the network, a first status message in response to determining that the first image processing operation is complete on the first medical image.

2. The method of claim 1, further comprising:
displaying the first status message on a display device of the user device;
transmitting, by the user device to the server over the network, a request to the server in response to displaying the first status message,
wherein the request adjusts one or more image processing parameters in the first image processing operation to produce an adjusted image processing operation; and
performing the adjusted image processing operation.

3. The method of claim 1, further comprising:
performing an artificial intelligence (AI) processing operation on a second medical image;
determining, by the server, whether the AI processing operation is complete on the second medical image; and
transmitting, by the server, a second status message in response to determining that the AI processing operation is complete on the second medical image.

4. The method of claim 3,
wherein the AI processing operation is an inference operation,
wherein the second medical image is an input to a machine-learning model, and
wherein the AI processing operation generates predicted medical data as an output of the machine-learning model.

5. The method of claim 3,
wherein the AI processing operation is a training operation for training a machine-learning model,
wherein the second medical image is a portion of a training dataset, and
wherein the machine-learning model is trained using the training dataset and a supervised learning algorithm.

6. The method of claim 1,
wherein the server is a medical image manager,
wherein the first image processing operation is performed by a processing server that is different from the medical image manager,
wherein the first status message corresponds to status information stored in a database in the medical image manager,
wherein the database comprises a patient table, a study table, a series table, and an image table, and
wherein the status information is disposed in a data field within the study table.

7. The method of claim 1,
wherein the user device is a hospital terminal,
wherein the hospital terminal accesses a medical image manager to obtain supplementary information regarding a first medical image, and
wherein the supplementary information comprises status information regarding an image processing operation at the medical image manager.

8. The method of claim 1,
wherein the first image processing operation is performed using an image compression function.

9. The method of claim 1,
wherein the medical imaging device is an X-ray radiography device.

10. The method of claim 1, further comprising:
determining, by the server, whether a second image processing operation performed an error based on a second medical image and an error detection function; and
transmitting, by the server to the user device over the network, a second status message identifying the error in response to determining that the second image processing operation performed the error.

11. The method of claim 1, further comprising:
selecting a second image among a plurality of images disposed in a processing queue,
wherein the second image is automatically selected based on a priority of the second image within the processing queue; and
performing a second image processing operation on the second image.

12. The method of claim 1,
wherein the first status message is displayed in a web browser on the user device.

13. A system, comprising:
a plurality of user devices;
a first processing server; and
a medical image manager coupled to the plurality of user devices and the first processing server over a network,
wherein the medical image manager comprises a computer processor, and
wherein the medical image manager is configured to perform a method comprising:
receiving, from a user device among the plurality of user devices and over the network, a first medical image that is generated using a medical imaging device;
determining whether a first image processing operation is complete on the first medical image; and
transmitting, to the user device over the network, a first status message in response to determining that the first image processing operation is complete on the first medical image.

14. The system of claim 13,
wherein the user device comprises a display device configured to display the first status message using a graphical user interface,
wherein the method further comprises adjusting one or more image processing parameters in the first image processing operation to produce an adjusted image processing operation, and
wherein the adjusted image processing operation is performed by the first processing server.

15. The system of claim 13, further comprising:
a second processing server that is coupled to the medical image manager over the network and configured to perform an artificial intelligence (AI) operation on a second medical image,
wherein the medical image manager is further configured to:
determine whether the AI processing operation is complete on the second medical image; and
transmit a second status message in response to determining that the AI processing operation is complete on the second medical image.

16. The system of claim 15,
wherein the AI processing operation is an inference operation,
wherein the second medical image is an input to a machine-learning model, and
wherein the AI processing operation generates predicted medical data as an output of the machine-learning model.

17. The system of claim 13,
wherein the first status message corresponds to status information stored in a database in the medical image manager,
wherein the database comprises a patient table, a study table, a series table, and an image table, and
wherein the status information is disposed in a data field within the study table.

18. The system of claim 13,
wherein the first status message is displayed in a web browser on the user device.

19. The system of claim 13,
wherein the medical imaging device is an X-ray radiography device.

20. A nontransitory computer readable medium comprising instructions, when executed by a computer, are configured to perform a method comprising:
receiving, from a user device over a network, a medical image that is generated using a medical imaging device;
determining whether an image processing operation is complete on the medical image, wherein the image processing operation is performed by a processing server; and
transmitting, to the user device over the network, a status message in response to determining that the image processing operation is complete on the medical image.
